(19) United States
(12) Patent Application Publication
(10) Pub. No.: US 2022/0411858 A1
(43) Pub. Date: Dec. 29, 2022

(54) RANDOM EMULSIFICATION DIGITAL ABSOLUTE QUANTITATIVE ANALYSIS METHOD AND DEVICE

(71) Applicant: MGI TECH CO., LTD., Shenzhen, Guangdong (CN)

(72) Inventors: Yun Xia, Shenzhen, Guangdong (CN); Xia Zhao, Shenzhen, Guangdong (CN); Yang Xi, Shenzhen, Guangdong (CN); Fang Chen, Shenzhen, Guangdong (CN); Hui Jiang, Shenzhen, Guangdong (CN)

(73) Assignee: MGI TECH CO., LTD., Shenzhen, Guangdong (CN)

(21) Appl. No.: 17/756,625

(22) PCT Filed: Nov. 29, 2019
(86) PCT No.: PCT/CN2019/122068

§ 371 Date: May 27, 2022

(51) Int. Cl. C12Q 1/6851

(52) U.S. Cl. CPC C12Q 1/6851

(57) ABSTRACT

A random emulsification digital absolute quantitative analysis method includes: performing random emulsification processing on a system to be emulsified to obtain several isolated reaction zones or droplets; determining the total number and volume information of the various reaction zones or droplets, the presence of target molecules to be tested in the respective reaction zones or droplets, and the number of reaction zones or droplets which do not contain the target molecules by combining acquired target images comprising image regions corresponding to the amplified reaction zones or droplets, and analyzing the target images; and accurately calculating the volume information of the various reaction zones or droplets, the presence of the target molecules to be tested in the respective reaction zones or droplets, and the number of reaction zones or droplets which do not contain the target molecules, the total number of target molecules in a sample to be tested.

### CROSS REFERENCE TO RELATED APPLICATIONS

This application is a U.S. National Phase Application of International Application No. PCT/CN2019/122068, filed Nov. 29, 2019, which is hereby incorporated by reference in its entirety.

### TECHNICAL FIELD

The present disclosure relates to the field of bioinformatic analysis, in particular to a random emulsification digital absolute quantitative analysis method and device.

### BACKGROUND

Accurate quantitative testing of a biochemical marker represented by nucleic acid or general biological or chemical substance molecules or particles and granules in other forms is of great significance for clinical diagnosis, progression monitoring and treatment of diseases, gene expression analysis, sequencing quality control and verification, microbiological test, and transgenic detection.

In the related art, equipment and instruments with digital polymerase chain reaction (digital PCR) functions are usually used to analyze a sample to be tested, so as to determine a concentration of nucleic acid molecules such as DNA or RNA in the sample to be tested. A general process of analyzing, by the equipment and instruments, the sample to be tested comprises: equally dividing a sample system with a certain volume to form several isolated reaction zones, and performing PCR amplification on each reaction zone at the same time, so as to only generate an amplified fluorescence signal (or other signals) in zones containing one or more target DNAs/RNAs before amplification, thereupon, calculating, through statistical analysis based on direct count or Poisson distribution principle, an initial copy number and concentration of the target DNAs/RNAs by acquiring a ratio of the number of the zones in which the amplified signal is generated to the number of all the zones and volumes of the respective zones. However, in the process of achieving the present disclosure, the inventor has found that in all absolute quantitative methods provided by the relevant equipment and instruments provided in the related art, equal probability distribution of sample molecules is achieved on the basis of zones with equal sizes. The dynamic range of the method in which the zones have equal sizes is severely limited to the total number of zones. Therefore, the relevant equipment and instruments provided in the related art can usually exert high sensitivity, high accuracy, good anti-interference performance, and other technical advantages in testing of low-concentration or low-abundance nucleic acid samples. For quantitative testing of samples at higher concentrations, it is generally necessary to perform gradient dilution on the samples for several times before zoning to obtain ideal response results, which cannot meet absolute quantitative requirements of nucleic acid samples at any concentration. In addition, the existing digital PCR products use a microfluidic technology and system to accurately divide fluids into nanoliter volumes or even femtoliter volumes to form uniformly sized zones or monodisperse droplets, leading to additional technical difficulty, operation difficulty, economy cost, and time cost for a user compared to real-time PCR.

### SUMMARY

The present disclosure aims to at least solve one of the technical problems in the related art to a certain extent.

In view of this, a first objective of the present disclosure is to provide a random emulsification digital absolute quantitative analysis method.

A second objective of the present disclosure is to provide a calculating method of simulating formation of a zone with any size or dispersed droplets with any volume for achieving digital absolute quantitative testing.

A third objective of the present disclosure is to provide a random emulsification digital absolute quantitative analysis device.

A fourth objective of the present disclosure is to provide a simulation system.

A fifth objective of the present disclosure is to provide an electronic device.

A sixth objective of the present disclosure is to provide a computer-readable storage medium.

In order to achieve the above objectives, an embodiment of the first aspect of the present disclosure provides a random emulsification digital absolute quantitative analysis method, comprising: performing random emulsification processing on a system to be emulsified in a preset container to obtain several isolated reaction zones or droplets, wherein the system to be emulsified comprises a sample to be tested; the total number of the reaction zones or droplets is randomly generated; the total number is a positive integer greater than 1; the reaction zones or the droplets are randomly generated; and a volume of each zone or droplet is randomly generated, and a sum of the volumes is not greater than the volume of the emulsified system; performing amplification processing on the reaction zones or droplets; acquiring, subsequent to that the amplification ends, images of the reaction zones or droplets to obtain a target image; analyzing image regions, corresponding to the respective reaction zones or droplets, in the target image to obtain volume information of the respective reaction zones or droplets, and determining presence of target molecules to be tested in the reaction zones or droplets; counting the number of reaction zones or droplets that do not contain the target molecules; determining, based on the total number of the reaction zones or droplets, the volume information of the respective reaction zones or droplets, the presence of the target molecules to be tested in the reaction zones or droplets and the number of the reaction zones or droplets that do not contain the target molecules, the total number of the target molecules in the sample to be tested.

The random emulsification digital absolute quantitative analysis method provided by the embodiment of the present disclosure comprises: performing random emulsification processing on a system to be emulsified in a preset container to obtain several isolated reaction zones or droplets, and causing amplification reaction in the reaction zones or droplets that contain target molecules to be tested; acquiring, subsequent to that the amplification ends, images of the amplified reaction zones or droplets to obtain a target image; analyzing image regions, corresponding to the respective reaction zones or droplets, in the target image to obtain volume information of the respective reaction zones or droplets, and determining presence of target molecules to be tested in the reaction zones or droplets; counting the number of reaction zones or droplets that do not contain the target molecules; determining, based on the total number of the reaction zones or droplets, the volume information of the respective reaction zones or droplets, the presence of the target molecules to be tested in the reaction zones or droplets and the number of the reaction zones or droplets that do not contain the target molecules, the total number of the target molecules in the sample to be tested. Thus, the total number of the target molecules in the sample to be tested is accurately calculated, which meets a requirement for an absolute quantitative analysis of a sample to be tested at any concentration.

In order to achieve the above objectives, an embodiment of the second aspect of the present disclosure provides a calculating method for achieving digital absolute quantitative testing by simulating formation of a zone with any size or dispersed droplets with any volume. The method is applied to a simulation system, and is characterized by comprising: setting the total number of target molecules to be m, wherein m is an integer greater than or equal to 0; setting the total number of reaction zones or droplets to be n, and randomly generating, based on the set total number n of the reaction zones or droplets, volume values νᵢ respectively corresponding to the n reaction zones or n droplets, wherein νᵢ represents a volume value of an i^{th} reaction zone or droplet, 1=1, 2, 3, . . . , n, wherein n is an integer greater than 1; calculating a total volume

$${\sum }_{i = 1}^{n} {v}_{i}$$

of the n reaction zones or droplets; randomly generating m groups of coordinate numerical value sets based on the total volume of the fluid system to be quantified, wherein a range of elements in the coordinate numerical value sets does not exceed the total volume of the fluid system to be quantified; representing, based on a dimension of each coordinate numerical value set, the volume value νᵢ of each reaction zone or droplet as n numerical value intervals which have the dimension and are connected according to a preset sequence; determining the number Xᵢ of coordinate numerical values contained in each of the n numerical value intervals; counting the total number of numerical value intervals containing zero coordinate numerical value (Xᵢ=0), and taking the obtained total number as the number C₀ of reaction zones or droplets that do not contain target molecules; determining, based on the total volume

$${\sum }_{i = 1}^{n} {v}_{i}$$

of the fluid system to be quantified, the total number n of the reaction zones or droplets, the volume value νᵢ of the respective reaction zones or droplets and the number C₀ of the reaction zones or droplets that do not contain the target molecules, an estimated value M of the total number of the target molecules; comparing whether the set total number m of the target molecules and the estimated value M of the total number of the target molecules are within a preset error range; and in response to being within the preset error range, determining that the simulation system is capable of performing calculation of digital absolute quantitative testing.

In order to achieve the above objectives, an embodiment of the third aspect of the present disclosure provides a random emulsification digital absolute quantitative analysis device, comprising: a random emulsification processing module configured to perform random emulsification processing on a system to be emulsified in a preset container to obtain several isolated reaction zones or droplets, wherein the system to be emulsified includes a sample to be tested; the total number of the reaction zones or droplets is randomly generated; the total number is a positive integer greater than 1; the reaction zones or the droplets are randomly generated; and a volume of each zone or droplet is randomly generated, and a sum of the volumes is not greater than the volume of the emulsified system; an amplification processing module configured to perform amplification processing on the reaction zones or droplets; an image acquisition module configured to, in response to detecting that the amplification ends, acquire images of the reaction zones or droplets to obtain a target image; an image analysis module configured to analyze image regions, corresponding to the respective reaction zones or droplets, in the target image to obtain volume information of the respective reaction zones or droplets, and determine presence of target molecules to be tested in the reaction zones or droplets; count the number of reaction zones or droplets that do not contain the target molecules; and a determination module configured to determine, based on the total number of the reaction zones or droplets, the volume information of the respective reaction zones or droplets, the presence of the target molecules to be tested in the reaction zones or droplets and the number of the reaction zones or droplets that do not contain the target molecules, the total number of the target molecules in the sample to be tested.

According to the random emulsification digital absolute quantitative analysis device provided by the embodiments of the present disclosure, random emulsification processing is performed on a system to be emulsified in a preset container to obtain several isolated reaction zones or droplets, and amplification processing is performed on the reaction zones or droplets; in response to that the amplification ends, images of the amplified reaction zones or droplets are acquired to obtain a target image; image regions, corresponding to the respective reaction zones or droplets, in the target image are analyzed to obtain volume information of the respective reaction zones or droplets, and presence of target molecules to be tested in the reaction zones or droplets is determined; the number of reaction zones or droplets that do not contain the target molecules is counted; and the total number of the target molecules in the sample to be tested is determined based on the total number of the reaction zones or droplets, the volume information of the respective reaction zones or droplets, the presence of the target molecules to be tested in the reaction zones or droplets and the number of the reaction zones or droplets that do not contain the target molecules. Thus, the total number of the target molecules in the sample to be tested is accurately calculated, and it is convenient to perform absolute quantitative analysis on a sample to be tested at any concentration.

In order to achieve the above objectives, an embodiment of the fourth aspect of the present disclosure provides a simulation system. The simulation system is configured to simulating formation of a zone with any size or dispersed droplets with any volume for achieving calculation of digital absolute quantitative testing. The simulation system comprises:

a first setting module configured to set the total number of target molecules to be m, wherein m is an integer greater than or equal to 0; a data generation module configured to set the total number of reaction zones or droplets to be n, and randomly generating, based on the set total number n of the reaction zones or droplets, volume values νᵢ (i=1, 2, 3, . . . , n) respectively corresponding to the n reaction zones or droplets, wherein n is an integer greater than 1; a first calculation module configured to calculate, based on the volume value vᵢ respectively corresponding to each of the n reaction zones or droplets, a total area or a total volume

$${\sum }_{i = 1}^{n} {v}_{i}$$

of a fluid system to be quantified; a generation module configured to randomly generate m groups of coordinate numerical value sets based on the total area or total volume

$${\sum }_{i = 1}^{n} {v}_{i}$$

of the fluid system to be quantified, wherein a range of elements in the coordinate numerical value sets does not exceed the total volume of the fluid system to be quantified; a representation module configured to represent, based on a dimension of each coordinate numerical value set, the area of each reaction zone or the volume value νᵢ of the droplet as n numerical value intervals which have the dimension and are connected according to a preset sequence; a first determination module configured to determine the number Xᵢ of coordinate numerical values contained in each of the n numerical value intervals; a counting module configured to count the total number of numerical value intervals containing zero coordinate numerical value, and taking the obtained total number as the number C₀ of reaction zones or droplets that do not contain target molecules; a second calculation module configured to calculate, based on the total volume

$${\sum }_{i = 1}^{n} {v}_{i}$$

of the fluid system to be quantified, the total number n of the reaction zones or droplets, the volume value vᵢ of the respective reaction zones or droplets and the number C₀ of the reaction zones or droplets that do not contain the target molecules, an estimated value M of the total number of the target molecules; and a verification module configured to compare whether the set total number m of the target molecules and the estimated value M of the total number of the target molecules are within a preset error range, and in response to being within the preset error range, determine that the simulation system is capable of performing the calculation of digital absolute quantitative testing.

In order to achieve the above objectives, an embodiment of the fifth aspect of the present disclosure provides an electronic device, comprising a memory, a processor, and a computer program that is stored in the memory and can be operated on the processor. The processor, when executing the program, implements the above-mentioned random emulsification digital absolute quantitative analysis method.

In order to achieve the above objectives, an embodiment of the sixth aspect of the present disclosure provides a computer-readable storage medium. When instructions stored in the storage medium are executed by a processor, the above-mentioned random emulsification digital absolute quantitative analysis method is implemented.

Additional aspects and advantages of the present disclosure will be provided in the following descriptions, part of which will become apparent from the following descriptions or be learned through the practice of the present disclosure.

### BRIEF DESCRIPTION OF DRAWINGS

The above and/or additional aspects and advantages of the present disclosure will become apparent and easily understandable from the following descriptions of the embodiments with reference to the accompanying drawings.

FIG. 1 is a schematic flowchart of a random emulsification digital absolute quantitative analysis method provided in an embodiment of the present disclosure;

FIG. 2 is a schematic diagram of target images of droplets that are acquired by a fluorescence microscope and photographed after random emulsification and amplification of DNA template molecules at different concentrations (diluted from 10⁻¹ to 10⁻⁶, a total of 6 concentrations), provided in an embodiment of the present disclosure;

FIG. 3 is a linear fitting result of quantitative data of the DNA template molecules at different concentrations, obtained after the target images in FIG. 2 are processed and analyzed, provided in an embodiment of the present disclosure.

FIG. 4 is a schematic flowchart of a calculating method for achieving digital absolute quantitative testing by simulating formation of a zone with any size or dispersed droplets with any volume, provided in an embodiment of the present disclosure;

FIG. 5 is a schematic diagram of a calculation principle of depicting a random emulsification and amplification model by simplifying it into a one-dimensional Poisson process.

FIG. 6 illustrates a logarithmic Gaussian distribution, and one-dimensional random simulation and calculation results, wherein the preset total number m of target molecules is 500, the number of zones or dispersed droplets is 256, a compliance mean of volumes is 4, and a variation coefficient is 0.001;

FIG. 7 illustrates a logarithmic Gaussian distribution, and one-dimensional random simulation and calculation results, wherein the preset total number m of target molecules is 500, the number of zones or dispersed droplets is 256, a compliance mean of volumes is 4, and a variation coefficient is 0.1;

FIG. 8 illustrates a logarithmic Gaussian distribution, and one-dimensional random simulation and calculation results, wherein the preset total number m of target molecules is 500, the number of zones or dispersed droplets is 256, a compliance mean of volumes is 4, and a variation coefficient is 10;

FIG. 9 illustrates a logarithmic Gaussian distribution, and one-dimensional random simulation and calculation results of dual target testing, wherein the preset total numbers m_{green} and mblue of target molecules are respectively 1000 and 25, the number of zones or dispersed droplets is 256, a compliance mean of volumes is 4, and a variation coefficient is 1;

FIG. 10 illustrates a logarithmic Gaussian distribution, wherein the preset total number m of target molecules is 1, 2, 5, 10, 20, 50, 100, 200, 500, 1000, 2000, 5000, 10000, 20000, 50000, and 100000, respectively, the number of zones or dispersed droplets is 256, a compliance mean of volumes is 4, and a variation coefficient is 0.001, 0.01, 0.1, 1, 10, and 100,respectively; and result statistics of one-dimensional random simulation and calculation, wherein each condition is performed for 500 times for repeated testing, and the influence of variation of volumes of the zones or dispersed droplets on calculation results is verified;

FIG. 11 is a graph depicting the statistical result obtained by a simulated calculation method using 2001000 Monte Carlo tests, and a logarithmic Gaussian distribution, wherein the number of zones or dispersed droplets is 256, a compliance mean of volumes is 4, and a variation coefficient is 1;

FIG. 12 is a structural schematic diagram of a random emulsification digital absolute quantitative analysis device provided in an embodiment of the present disclosure;

FIG. 13 is a structural schematic diagram of a random emulsification digital absolute quantitative analysis device provided in another embodiment of the present disclosure;

FIG. 14 is a structural schematic diagram of a simulation system provided in an embodiment of the present disclosure; and

FIG. 15 is a structural schematic diagram of an electronic device provided in an embodiment of the present disclosure.

### DESCRIPTION OF EMBODIMENTS

The embodiments of the present disclosure are described in detail below. Examples of the embodiments are shown in the accompanying drawings. The same or similar reference numerals represent the same or similar elements or elements having the same or similar functions throughout. The embodiments described below with reference to the drawings are exemplary, and are intended to explain the present disclosure, and should not be construed as limiting the present disclosure.

A random emulsification digital absolute quantitative analysis method and device of the embodiments of the present disclosure are described below with reference to the accompanying drawings.

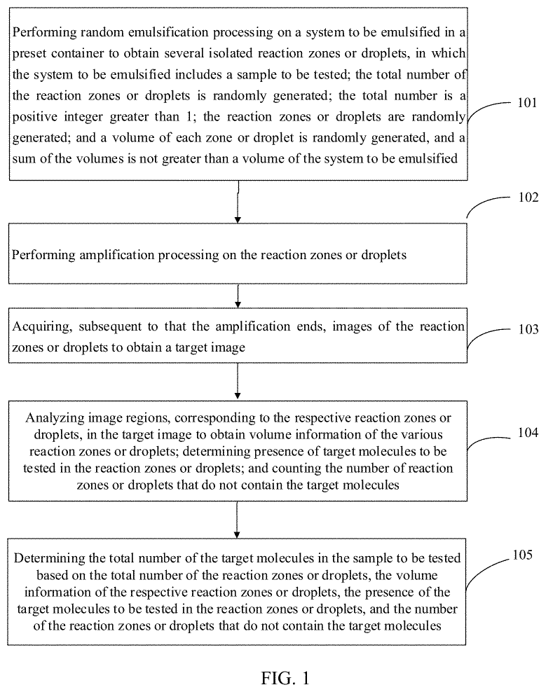

FIG. 1 is a schematic flowchart of a random emulsification digital absolute quantitative analysis method provided in an embodiment of the present disclosure.

As shown in FIG. 1, the random emulsification digital absolute quantitative analysis method may include the following steps.

In step 101, random emulsification processing is performed on a system to be emulsified in a preset container to obtain several isolated reaction zones or droplets, wherein the system to be emulsified includes a sample to be tested; the total number of the reaction zones or droplets is randomly generated; the total number is a positive integer greater than 1; the reaction zones or the droplets are randomly generated; and a volume of each zone or droplet is randomly generated, and a sum of the volumes is not greater than the volume of the system to be emulsified.

It should be noted that an execution main body of the random emulsification digital absolute quantitative analysis method is a random emulsification digital absolute quantitative analysis device. The random emulsification digital absolute quantitative analysis device can be configured in an electronic device.

The electronic device in this embodiment is an electronic device with a random emulsification digital absolute quantitative analysis function. The electronic device can accurately calculate the total number of target molecules in the sample to be tested by the random emulsification digital absolute quantitative analysis method in the device.

The above preset container may include, but is not limited to, a flat rectangular capillary tube, a double-sided glass closed sandwich pool or a glass-monocrystalline silicon closed sandwich pool, and other containers, so that the random emulsification system to be quantified forms a quasi-two-dimensional or two-dimensional droplet array.

It can be understood that the reaction zones or droplets in this embodiment are randomly formed, which means that the sizes of the reaction zones and droplets in this embodiment are random, that is, the volume size of the reaction zones or droplets is random.

The zone with any size or the dispersed droplets with any volume in the present disclosure means that a fluid system to be quantified is divided into several isolated reaction zones or droplets with smaller volumes. The “several” here can be a natural number with a given certain numerical value, or can be a random number or variable without a given numerical value. The numerical value of the smaller volume described here can be unconstrained and unrestricted by any conditions and rules, including all possible divided results. For example, the smaller volume can be set to be a certain constant (such as dividing 1 μL into 1000 pieces of 1 nLs), or can be set to be a plurality of constants (such as dividing 1 μL into 100 pieces of each of 1 nLs, 2 nLs, 3 nLs and 4 nLs). An interval or ratio of the plurality of constants can be constant or random, or can be set to be a generalized random number or variable with a certain discrete or continuous distribution function (such as dividing 1 μL into random numerical value volumes with uniform distribution or Gaussian distribution from 1 nL to 5 nL), and can also be set to any variable that only needs to satisfy the limit of the total volume (such as dividing 1 μL into X₁nL, X₂nL, . . . , XₙnL, wherein X₁+X₂+ . . . +Xₙ=1000, and X₁, X₂, . . . , Xₙ≥0).

It can be understood that, in this embodiment, in the formed reaction zones or droplets, some reaction zones or droplets do not contain target molecules, while other zones contain one or more target molecules. Or, some reaction zones or droplets contain a certain number of target molecules, while other zones contain another certain number of target molecules.

It should be noted that, in addition to the sample to be detected, the system to be emulsified in this embodiment may also include a preset amplification system, a preset continuous phase fluid, and a corresponding surfactant.

The preset amplification system may include, but is not limited to, polymerase chain reaction (PCR), loop-mediated isothermal amplification (LAMP), helicase-dependent amplification (HDA), recombinase polymerase amplification (RPA), strand displacement amplification (SDA), and other different amplification systems.

The target molecule in this embodiment may be described by taking a biomolecule represented by a nucleic acid molecule as an example.

It can be understood that the target molecule in this embodiment may also be other types of biomolecules. For example, the target molecule may be a protein, which is not limited in this implementation.

It should be understood that the target molecule in this embodiment can be not only a biomolecule, but also a chemical substance molecule. For example, the target molecule can be a metal ion. A specific process of calculating the content of the metal ion is similar to that the random emulsification digital absolute quantitative analysis method disclosed by the present disclosure, and will not be repeated here.

The preset amplification system is an amplification system preset by a user in the electronic device according to the target molecules, so as to satisfy the purpose of the user for adjusting, according to the target molecules, the amplification system.

The preset continuous phase fluid may include, but is not limited to, a carbon base, a silicon base, fluorinated oil, and the like.

In step 102, amplification processing is performed on the reaction zones or droplets.

Specifically, after several reaction zones or droplets with random sizes are formed, the amplification processing can be performed on all of the reaction zones or droplets simultaneously.

It can be understood that when the amplification processing is performed on the reaction zones or droplets, in the reaction zones or droplets containing the target molecules, specific primers will lead to temperature-sensitive cyclic amplification of the target molecules under the efficient catalysis action of nucleic acid polymerase, thus amplifying signal of a target molecule to be tested, and thereby enhancing signals of indicators in the corresponding zones or droplets. However, reaction zones or droplets that do not contain the target molecules will not lead to an enhanced indicator signal caused by the amplification reaction, so that it can be determined, based on different enhancement states of the indicator signals, that each zone or droplet contains or does not contain the target molecules.

The indicator may include, but is not limited to, a fluorescent agent.

For example, a nucleic acid molecule serving as the target molecule is taken as an example. In the reaction zone or droplet that contains the nucleic acid molecule, DNA of the nucleic acid is subjected to temperature-sensitive cyclic amplification using specific primer under the efficient catalysis of the nucleic acid polymerase, so that a biomolecule signal of a gene or nucleic acid fragment to be tested is subjected to exponential amplification, and the fluorescence quantum yield of a specific dye molecule in the corresponding amplification system will also be amplified, that is, the intensity of a fluorescence signal will be increased.

In step 103, in response to that the amplification ends, images of the reaction zones or droplets are acquired to obtain a target image.

In this embodiment, the preset amplification system includes a preset indicator. When the reaction zones or droplets are amplified, whether the amplification processing ends is determined based on the intensity of an indication signal of the preset indicator. When detecting the intensity of the indication signal of the preset indicator no longer change, it is determined that the amplification processing ends.

In this embodiment, in order to facilitate the subsequent acquisition of volume information of the respective zones or droplets on the basis of the acquired images, the amplified droplets can also be subjected to squeezing deformation processing before the images of the reaction zones or droplets are acquired to obtain the target image.

As an exemplary implementation, after the amplification processing is performed on the reaction zones or droplets, the amplified droplets in the preset container can be subjected to appropriate squeezing deformation processing, and the images of the reaction zones or droplets in the preset container are acquired through an image acquisition module, to obtain the target image.

The image acquisition module includes a camera (a charge-coupled device (CCD) image sensor or a complementary metal-oxide-semiconductor (CMOS) image sensor), an excitation light source, a lens group, a beam splitter, a filter module, etc.

The images of all reaction zones or droplets can be acquired by the camera, so that the target image includes image regions corresponding to the respective reaction zones or droplets.

In step 104, image regions, corresponding to the respective reaction zones or droplets, in the target image are analyzed to obtain volume information of the respective reaction zones or droplets; presence of target molecules to be tested in the reaction zones or droplets is determined; and the number of reaction zones or droplets that do not contain the target molecules is counted.

After the target image is acquired, the image region of each reaction zone or droplet in the target image can be determined, and the volume information of each reaction zone or droplet is calculated according to position information of the image region of each reaction zone or droplet in the target image.

In this embodiment, a specific implementation process of analyzing the image regions, corresponding to the respective reaction zones or droplets, in the target image to obtain the number of reaction zones or droplets that do not contain the target molecules is as follows: extracting features of the image regions, corresponding to the respective reaction zones or droplets, in the target image, so as to obtain feature information corresponding to each image region; for each image region, matching the feature information of the image region with preset feature information; in response to that the feature information of the image region is not matched with the preset feature information, determining that the reaction zone or droplet corresponding to the image region does not contain the target molecules; and determining the total number of image regions, which are not matched with the preset feature information, in the target image, and taking the total number of the image regions as the number of the reaction zones or droplets that do not contain the target molecules.

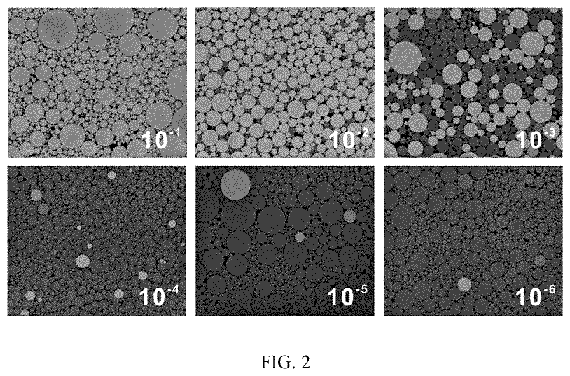

The images of the droplets after the amplification processing in a sequencing flow pool are acquired by the camera. The schematic diagram of the acquired target image is as shown in FIG. 2.

It should be noted that after the target image is acquired, it can be determined, according to the features of the images in the image regions, corresponding to the respective reaction zones or droplets, in the target image, whether the image regions corresponding to the respective reaction zones or droplets contain the target molecules. For example, bright droplet regions in the target image contain the target molecules, and dark droplet regions do not contain the target molecules.

In step 105, the total number of the target molecules in the sample to be tested is determined based on the total number of the reaction zones or droplets, the volume information of the respective reaction zones or droplets, the presence of the target molecules to be tested in the reaction zones or droplets and the number of the reaction zones or droplets that do not contain the target molecules.

In this embodiment, the number of the target molecules in the respective reaction zones or droplets complies with the Poisson distribution of independently non-identical distribution, and the number of the reaction zones or droplets that do not contain the target molecules complies with the Poisson binomial distribution. The total number of the target molecules in the sample to be tested is determined according to the following formula:

$${\sum }_{p = 1}^{n - j} \frac{{v}_{p} \times {e}^{- {mv}_{p} / {\sum }_{i = 1}^{n} {v}_{i}}}{{\sum }_{i = 1}^{n} {v}_{i} \times ( 1 - {e}^{- {mv}_{p} / {\sum }_{i = 1}^{n} {v}_{i}} )} = {\sum }_{q = 1}^{j} ( {v}_{q} / {\sum }_{i = 1}^{n} {v}_{i} )$$

wherein m represents the total number of the target molecules to be determined in the emulsified system; n represents the total number of the reaction zones or droplets; j represents a value of the number C₀ of the reaction zones or droplets that do not contain the target molecules; νᵢ(i=1, 2 , 3, . . . , n) represents the volume of an i^{th} reaction zone or droplet; νₚ(p=1, 2, 3, . . . , n−j) represents the volume of a p^{th} reaction zone or droplet that contains the target molecules; v (q =1, 2, 3, . . . , j) represents the volume of a q^{th} reaction zone or droplet that does not contain the target molecules; and e is a natural constant. As described above, n, j, νᵢ, νₚ, and ν_{q} are all determined or statistically obtained by analyzing the target image.

Specifically, the process of distributing the target molecules in a bulk solution to several multivolume droplet systems can be regarded as a series of independently non-identical distribution Bernoulli trials. For a reaction zone or droplet with a volume of νᵢ, a probability that the total number Xᵢ of the target molecules contained in the reaction zone or droplet is k (k is a non-negative integer) is:

$$P \{ {X}_{i} = k \} = ( \begin{matrix}m \\ k\end{matrix} ) \times {( {v}_{i} / {\sum }_{i = 1}^{n} {v}_{i} )}^{k} \times {( 1 - {v}_{i} / {\sum }_{i = 1}^{n} {v}_{i} )}^{m - k} , \qquad ( 1 )$$

That is, Xᵢ complies with a binomial distribution, wherein

$$( \begin{matrix}m \\ k\end{matrix} )$$

is a combination number of k molecules randomly selected from the total number m of target molecules, and

$${v}_{i} / {\sum }_{i = 1}^{n} {v}_{i}$$

is the probability of distributing a single target molecule to this reaction zone or droplet. When the total number m of the target molecules is an undetermined constant, a mathematical expectation

$$m {v}_{i} / {\sum }_{i = 1}^{n} {v}_{i}$$

of the total number Xᵢ of the target molecules contained in the droplet is also a constant. At the same time, if the probability

$${v}_{i} / {\sum }_{i = 1}^{n} {v}_{i}$$

is small enough, Xᵢ approximately complies with the Poisson distribution of

$${λ}_{i} = m {v}_{i} / {\sum }_{i = 1}^{n} {v}_{i} :$$

$$P \{ {X}_{i} = k \} = \frac{{λ}_{i}^{k} \times {e}^{- {λ}_{i}}}{k !} = \frac{{( m {v}_{i} / {\sum }_{i = 1}^{n} {v}_{i} )}^{k} \times {e}^{- m {v}_{i} / {\sum }_{i = 1}^{n} {v}_{i}}}{k !} , \qquad ( 2 )$$

Particularly, in case of k=0, a probability that the reaction zones or droplets do not contain the target molecules is:

$$P \{ {X}_{i} = 0 \} = {e}^{- {mv}_{i} / {\sum }_{i = 1}^{n} {v}_{i}} , \qquad ( 3 )$$

Correspondingly, in case of k≥1, a probability that the reaction zones or droplets contain the target molecules is:

$$P \{ {X}_{i} \geq 1 \} = 1 - {e}^{- {mv}_{i} / {\sum }_{i = 1}^{n} {v}_{i}} , \qquad ( 4 )$$

Further, a probability that the number C₀ of the reaction zones or droplets that do not contain the target molecules is j is:

$$P \{ {C}_{0} = j \} = {\sum }_{s = 1}^{( \begin{matrix}n \\ j\end{matrix} )} ( {\prod }_{p = 1}^{n - j} P \{ {X}_{p} \geq 1 \} \times {\prod }_{q = 1}^{j} P \{ {X}_{q} = 0 \} ) , \qquad ( 5 )$$

$$( \begin{matrix}n \\ j\end{matrix} )$$

that is, C₀ complies with the Poisson binomial distribution, wherein is a combination number of j reaction zones or droplets that do not contain the target molecules and are randomly selected from the total number n of the reaction zones or droplets. Thus, the volume of the reaction zone or droplet that does not contain the target molecules can be calculated as ν_{q} (q=1, 2, 3, . . . , j), respectively, and a conditional probability that the number

C₀ of the reaction zones or droplets that do not contain the target molecules is j is:

$$P \{ {C}_{0} = j \vert {\sum }_{q = 1}^{j} {X}_{q} = 0 \} = {\prod }_{p = 1}^{n - j} ( 1 - {e}^{- {mv}_{p} / {\sum }_{i = 1}^{n} {v}_{i}} ) \times {\prod }_{q = 1}^{j} {e}^{- m {v}_{q} / {\sum }_{i = 1}^{n} {v}_{i}} , \qquad ( 6 )$$

According to the maximum likelihood estimation method, the formula that needs to be satisfied when the conditional probability is maximized can be derived according to formula (6):

$${\sum }_{p = 1}^{n - j} \frac{{v}_{p} \times {e}^{- {mv}_{p} / {\sum }_{i = 1}^{n} {v}_{i}}}{{\sum }_{i = 1}^{n} {v}_{i} \times ( 1 - {e}^{- {mv}_{p} / {\sum }_{i = 1}^{n} {v}_{i}} )} = {\sum }_{q = 1}^{j} ( {v}_{q} / {\sum }_{i = 1}^{n} {v}_{i} ) , \qquad ( 7 )$$

Since n, j, νᵢ, νₚ, and v_{q} on the left and right ends of formula (7) are all determined or statistically obtained by analyzing the target image, which are known numbers, formula (7) is transformed into an equation only containing a unique unknown number, m. Therefore, an optimal value of m can be calculated using the interval dichotomy, the Newton iteration method, the secant method, the Newton interpolation method, and the like to make the left and right ends of formula (7) equal. This value is the total number of the target molecules to be determined contained in the emulsification system.

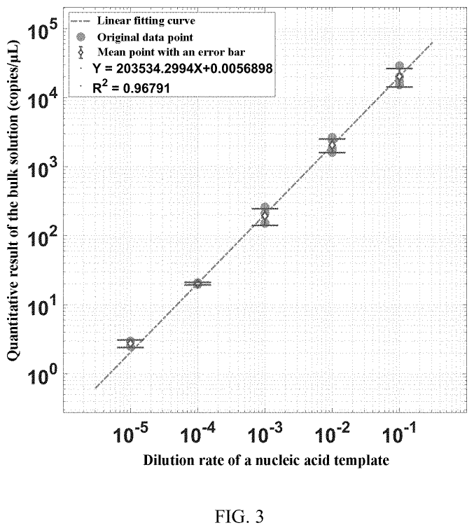

FIG. 3 is a linear fitting result of quantitative data of DNA template molecules at different concentrations, obtained after the acquired target images in FIG. 2 are processed and analyzed by the above calculation method provided in an embodiment of the present disclosure.

In this embodiment, in addition to determining the total number of the target molecules in the sample to be detected by the above formulas, a pre-established statistical analysis model used for determining the number of target molecules can also be used to determine the total number of the target molecules in the sample to be detected.

Specifically, the total number of the zones or droplets, the volume information of the respective reaction zones or droplets and the number of the reaction zones or droplets that do not contain the target molecules are input into a pre-established analysis model. An output of the analysis model is the total number of the target molecules in the sample to be tested.

The pre-established analysis model has learned a mapping relationship with the target molecules according to the total number of the zones or droplets, the volume information of the respective reaction zones or droplets and the number of the reaction zones or droplets that do not contain the target molecules.

In this embodiment, after the total number of the target molecules in the sample to be tested is determined, the concentration of the corresponding target molecules can also be obtained by further calculation.

Specifically, the concentration of the target molecules in the sample to be detected can be determined based on the total number of the target molecules in the sample to be tested and the volume information of the sample to be tested.

According to the random emulsification digital absolute quantitative analysis method provided by the embodiment of the present disclosure, random emulsification processing is performed on a system to be emulsified in a preset container to obtain several isolated reaction zones or droplets, and amplification processing is performed on the reaction zones or droplets; in response to that the amplification ends, images of the amplified reaction zones or droplets are acquired to obtain a target image; image regions, corresponding to the respective reaction zones or droplets, in the target image are analyzed to obtain volume information of the respective reaction zones or droplets, and presence of target molecules to be tested in the reaction zones or droplets is determined; the number of reaction zones or droplets that do not contain the target molecules is counted; and the total number of the target molecules in the sample to be tested is determined based on the total number of the reaction zones or droplets, the volume information of the respective reaction zones or droplets, the presence of the target molecules to be tested in the reaction zones or droplets and the number of the reaction zones or droplets that do not contain the target molecules. Thus, the total number of the target molecules in the sample to be tested is accurately calculated, which meets a requirement for an absolute quantitative analysis of a sample to be tested at any concentration.

In this embodiment, in order to verify the feasibility of the above method for calculating the total number of the target molecules, this embodiment also provides a calculation method of simulating formation of a zone with any size or dispersed droplets with any volume for achieving digital absolute quantitative testing. The method is applied to the simulation system. It should be noted that in the present invention, the zone with any size or the dispersed droplets with any volume means that a fluid system to be quantified is divided into several isolated reaction zones or droplets with smaller volumes.

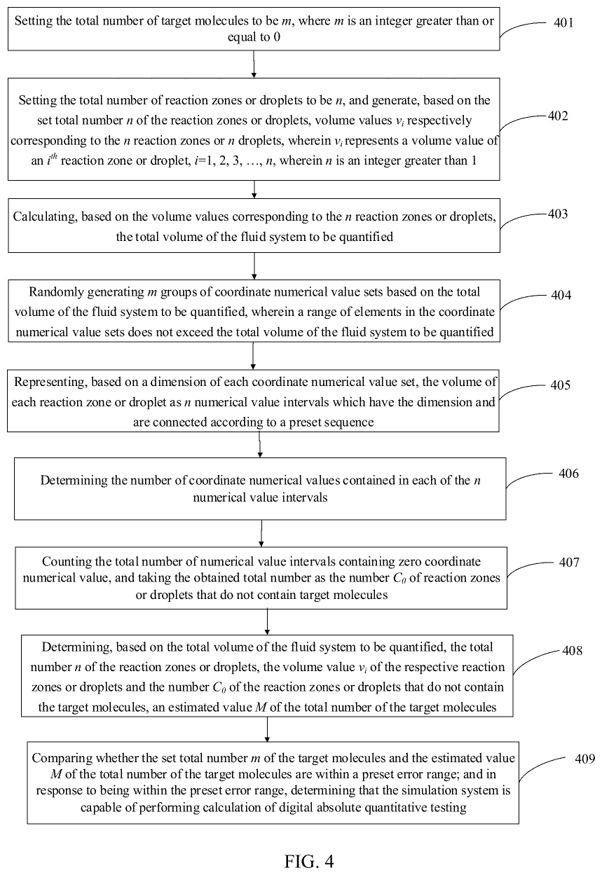

FIG. 4 is a schematic flowchart of a calculating method for achieving digital absolute quantitative testing by simulating formation of a zone with any size or dispersed droplets with any volume, provided in an embodiment of the present disclosure.

As shown in FIG. 4, the method may include the following steps.

In step 401, the total number of target molecules is set to be m, wherein m is an integer greater than or equal to 0.

According to a setting method, the total number can be set to be certain constant, or can be set to be a variable within a certain range through a certain function. At each simulation, the variable is set to be a certain constant. At the end, the variable is reset.

In step 402, the total number of reaction zones or droplets is set to ben, and volume values vᵢ respectively corresponding to the n reaction zones or n droplets are generated based on the set total number n of the reaction zones or droplets, wherein vᵢ represents a volume value of an i^{th} reaction zone or droplet, i=1, 2, 3, . . . , n, wherein n is an integer greater than 1.

A sum of the volumes of all the formed reaction zones or droplets is equal to the total volume of the fluid system to be quantified.

The setting method may be implemented by determining a numerical value to be set as a certain constant or several constants, or may be implemented in a simulation terminal by using a certain random number or variable generator of a certain discrete or continuous distribution function, or may be implemented by any combination of two methods.

In one embodiment of the present disclosure, the volumes of the above reaction zones or droplets can also conform to a certain distribution rule. As one exemplary implementation, a user can also set parameter information of a preset distribution with which the volumes of the reaction zones or droplets comply.

Correspondingly, the step that volume values respectively corresponding to the n reaction zones or n droplets are generated based on the set total number n of the reaction zones or droplets includes: volume values respectively corresponding to the n reaction zones or droplets are generated based on the parameter information of the preset distribution and the set total number n of the reaction zones or droplets.

In this embodiment, the preset distribution includes a logarithmic Gaussian distribution, and the parameter information includes a mean, a standard deviation, and a variation coefficient.

Of course, the above preset distribution may also be other distributions. For example, the preset distribution is a uniform distribution. The total volume of the fluid system to be quantified is 1 μL. When a plurality of droplets with different volumes are formed, the total volume of the fluid system to be quantified is divided into 100 pieces of each of 1 nL, 2 nL, 3 nL, and 4 nL, so that there are a total of 400 droplets with multiple volumes.

In step 403, the total volume of the fluid system to be quantified is calculated based on the volume values respectively corresponding to the n reaction zones or droplets.

In step 404, m groups of coordinate numerical value sets are randomly generated based on the total volume of the fluid system to be quantified, wherein a range of elements in the coordinate numerical value sets does not exceed the total volume of the fluid system to be quantified.

The dimension of the coordinate numerical value set may be one-dimensional, two-dimensional or three-dimensional, and not be limited in this embodiment.

It should be noted that in this embodiment, the simulation is described by taking a one-dimensional coordinate numerical value set as an example.

In this embodiment, a random number or variable generator can be used to generate the m groups of coordinate numerical value sets that satisfy a specific distribution. The specific distribution that the coordinate numerical value sets satisfy may be uniform distribution, Gaussian distribution, logarithmic Gaussian distribution, and the like.

In step 405, the volume value of each reaction zone or droplet is represented, based on a dimension of each coordinate numerical value set, as n numerical value intervals which have the dimension and are connected according to a preset sequence.

That is, the volume value of each zone or dispersed droplet is represented as n numerical value intervals which have the dimension and are connected according to a preset sequence. (For example, in the case of one dimension: the numerical value intervals can be connected in the sequence according to the serial numbers i of vᵢ, i=1, 2, 3, . . . , n. For example, the interval with a length of v₁ is on the left of the interval with a length of v₂; the interval with a length of v₃ is on the right of the interval with the length of v₂; by parity of reasoning, the length of the rightmost interval is vₙ).

In step 406, the number of coordinate numerical values contained in each of the n numerical value intervals is determined.

Based on the number Xᵢ of the coordinate numerical values contained in the numerical value interval represented by the volume of each zone or dispersed droplet, i.e., corresponding to the number of molecules contained in the volume of the zone or dispersed droplet, 0 molecule, 1 molecule, 2 molecules, . . . , as high as the number Cₖ of the zones or dispersed droplets with the preset total number m of target molecules in step 401, k=0, 1, 2, . . . , m is (are) counted, respectively.

In step 407, the total number of numerical value intervals containing zero coordinate numerical value is counted, and the obtained total number is taken as the number C₀ of reaction zones or droplets that do not contain target molecules.

In step 408, an estimated value M of the total number of the target molecules is determined based on the total volume

$${\sum }_{i = 1}^{n} {v}_{i}$$

of the fluid system to be quantified, the total number n of the reaction zones or droplets, the volume value vᵢ of the respective reaction zones or droplets and the number C₀ of the reaction zones or droplets that do not contain the target molecules.

In step 409, it is compared whether the set total number m of the target molecules and the estimated value M of the total number of the target molecules are within a preset error range; and in response to being within the preset error range, it is determined that the simulation system is capable of performing the calculation of digital absolute quantitative testing.

According to a random simulation method for achieving digital absolute quantitative testing by simulating formation of a zone with any size or dispersed droplets with any volume in the embodiment of the present disclosure, a digital absolute quantitative amplification experiment is designed and performed, and numerical values of the sizes of the respective zones or droplets are analyzed and acquired based on experimental data, such as the volume vᵢ of each zone or droplet, and the total number n of the zones or droplets. At the same time, the amplification reaction leads to amplified amplification signals in the zones or droplets containing the target molecules, while does not lead to any amplified amplification signals in the zones or droplets that do not contain the target molecules, so that it is determined, based on different reaction states, that each zone or droplet contains the target molecules or does not contain the target molecules, and the total number C₀ of the zones or droplets that do not contain the target molecules is statistically obtained. The simulation system is used below to perform the calculation of the absolute quantitative testing:

It is set that the preset total number m of the target molecules is each integer in a large enough number of integer numerical value (assumingM) intervals that causes all the zones or dispersed droplets to at least contain 1 target molecule, and the minimum integer in the interval is 0, and it is set that the numerical values of the sizes (areas or volumes) of the n simulated zones or dispersed droplets are equal to the numerical values, which are analyzed and acquired based on the experimental data, of the sizes of the respective zones or droplets, such as the volume vᵢ of each zone or droplet. Whenever m is set as an integer numerical value, steps 401 to 407 of the random simulation method are repeated for several times. The number R of repetition is a sufficiently large numerical value with statistical significance, such as 500, 1000, 10000, etc., and the specific number of R can be properly adjusted according to the calculation accuracy and the calculation overhead. Each time when a random simulation experiment is completed, a corresponding C₀ result (minimum: 0, maximum: n) can be obtained, and a pair of corresponding preset m and C₀ numerical values can be obtained. When all R×(M+1) random simulation experiments with a total of (M+1) preset numerical values of m from 0 to M are completed, a total of R×(M+1) pairs of preset m and C₀ numerical values can be obtained. The preset m values corresponding to the same C₀ value are counted in different classes, and the frequency and probability of each m value are calculated. Furthermore, a probability density function ƒ(x) of the m value is obtained by fitting or interpolation, thereby calculating a mathematical expectation E(m) and variance D(m) of each m value corresponding to the C₀ value. An observed value of the total number C₀ of the zones or droplets that do not contain the target molecules obtained by determining, through the amplification reaction, the reaction state of each zone or droplet, and the simulated probability density function ƒ(x) of the m value corresponding to C₀ are subjected to comparative analysis to obtain a calculation result E(m) of the simulation calculation method and a corresponding m value confidence interval [mₘᵢₙ, mₘₐₓ].

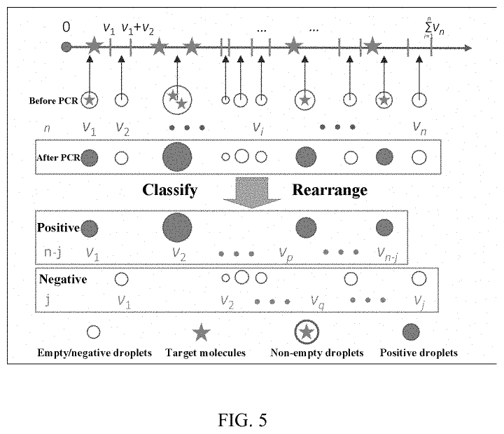

It should be noted that in this embodiment, the subsequently described embodiments of verifying the random simulation method are all described by taking a droplet as an example. A calculation principle of depicting a random emulsification and amplification model is simplified into a one-dimensional Poisson process, as shown in FIG. 5.

Embodiment 1 of verifying the random simulation method: This embodiment is used for verifying the feasibility of using the random simulation method to perform the simulation of a random emulsification zoning, and evaluating the influence of the variation of the volumes of the zones on an absolute quantitative result.

1. The number m of molecules is preset to be 500 to simulate the case that the number of target molecules in the system is 500.

2. The number n of dispersed droplets generated by the random emulsification is preset to be 256, and the logarithmic Gaussian distribution is set (the volumes of the droplets generated by random emulsification in general case all satisfy the logarithmic Gaussian distribution), wherein the compliance mean of the volumes νᵢ of the dispersed droplets is 4. The standard deviations of the volumes of the dispersed droplets are respectively set to be 0.004, 0.04, 0.4, 4, 40, and 400, and the corresponding variation coefficients are respectively 0.001, 0.01, 0.1, 1, 10, and 100. 256 volume numerical values are randomly generated according to the various determined parameters.

3. The sum

$${\sum }_{i = 1}^{n} {v}_{i}$$

of the volumes of the 256 dispersed droplets is calculated, and 500 numerical value points are randomly generated at the interval of

$$[ \begin{matrix}0 , & {\sum }_{i = 1}^{n} {v}_{i} ]\end{matrix} ;$$

according to sub-intervals

$$[ {\sum }_{t = 1}^{i} {v}_{t} - {v}_{i} , {\sum }_{t = 1}^{i} {v}_{t} ]$$

corresponding to the 256 dispersed droplets in the interval

$$[ \begin{matrix}0 , & {\sum }_{i = 1}^{n} {v}_{i} ]\end{matrix} ,$$

distribution of the 500 numerical value points in each sub-interval is determined; the number X, of the numerical value points contained in each sub-interval is counted, respectively; and the number Cₖ of the dispersed droplets containing k (k=0, 1, 2, . . . , m) numerical value points is counted.

4. The estimated valueM of the total number of the target molecules in all dispersed droplets is calculated according to C₀ and the above analysis and calculation method. The estimated values M of the total number of the target molecules is 516.8, 518.15, 507.9, 526.4, 493.7 and 522.05 when the variation coefficients of the volumes of the dispersed droplets are 0.001, 0.01, 0.1, 1, 10 and 100, respectively.

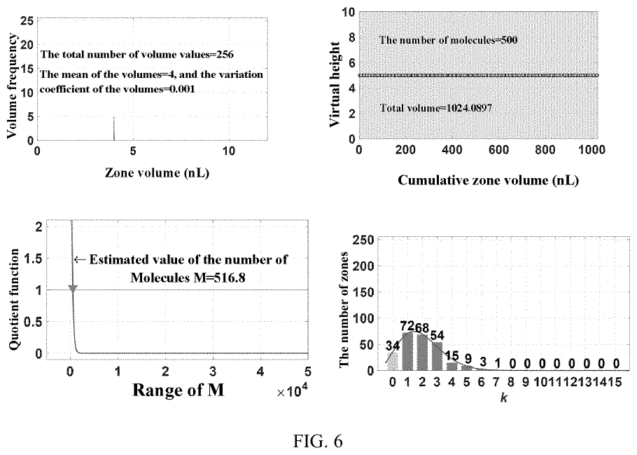

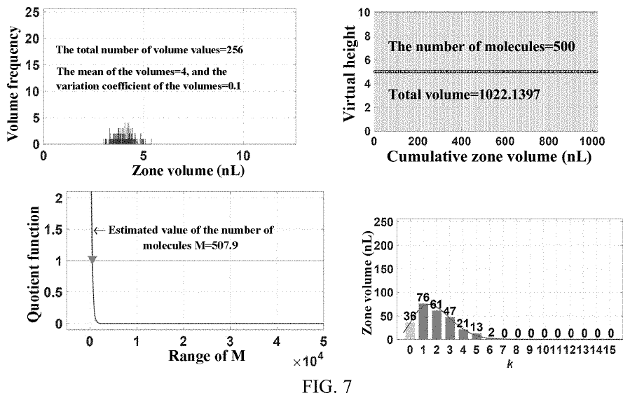

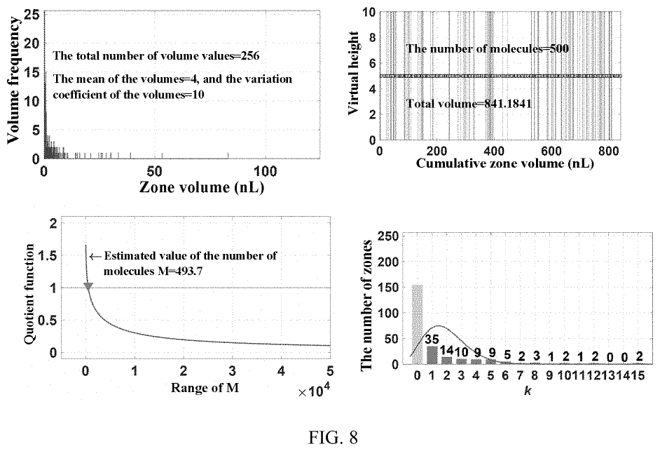

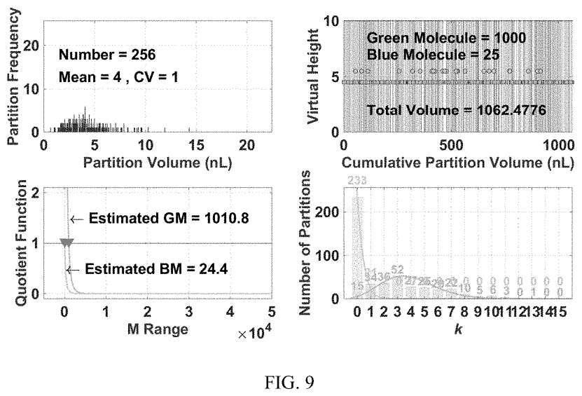

Visualization results of the simulation experiments are shown in FIGS. 6 to 9. FIG. 6 illustrates simulation and calculation visualization results when the variation coefficient is 0.001, wherein the total volume of the dispersed droplets is 1024.0897, C₀ is 34, and the estimated value M is 516.8. FIG. 7 illustrates simulation and calculation visualization results when the variation coefficient is 0.1, wherein the total volume of the dispersed droplets is 1022.1397, C₀ is 36, and the estimated value M is 507.9. FIG. 8 illustrates simulation and calculation visualization results when the variation coefficient is 10, wherein the total volume of the dispersed droplets is 841.1841, C₀ is 154, and the estimated value M is 493.7. FIG. 9 illustrates simulation and calculation visualization results obtained by performing the random emulsification on two kinds of target molecules when the variation coefficient is 0.1, wherein the total numbers of the two kinds of target molecules are respectively 25 and 1000; the preset total volume of the dispersed droplets is 1062.4776; C₀ of target molecules labeled with green is 233, and the estimated valueMis 24.4; and C₀ of target molecules 2 labeled with blue is 15, and the estimated valueM is 1010.8.

Embodiment 2 of evaluating the influence of the variation of the volumes of the zones on the absolute quantitative results: In this embodiment, the random simulation method of the present disclosure is used to evaluate the influence of the variation of the volumes of the dispersed droplets generated by the random emulsification on the absolute quantitative results.

1. The number m of molecules is preset to be 1, 2, 5, 10, 20, 50, 100, 200, 500, 1000, 2000, 5000, 10000, 20000, 50000, and 100000, respectively.

2. The number n of dispersed droplets generated by the random emulsification is preset to be 256, and the logarithmic Gaussian distribution is set (the volumes of the droplets generated by random emulsification in general case all satisfy the logarithmic Gaussian distribution), wherein the compliance mean of the volumes νᵢ of the dispersed droplets is 4. The standard deviations of the volumes of the dispersed droplets are respectively set to be 0.004, 0.04, 0.4, 4, 40, and 400, and the corresponding variation coefficients are respectively 0.001, 0.01, 0.1, 1, 10, and 100. 256 volume numerical values are randomly generated according to the various determined parameters.

3. The sum

$${\sum }_{i = 1}^{n} {v}_{i}$$

of the volumes of the 256 dispersed droplets is calculated, and the above m numerical value points are randomly generated at the interval of

$$[ \begin{matrix}0 , & {\sum }_{i = 1}^{n} {v}_{i} ] ;\end{matrix}$$

according to sub-intervals

$$[ {\sum }_{t = 1}^{i} {v}_{t} - {v}_{i} , {\sum }_{t = 1}^{i} {v}_{t} ]$$

corresponding to the 256 dispersed droplets in the interval

$$[ \begin{matrix}0 , & {\sum }_{i = 1}^{n} {v}_{i} ] ,\end{matrix}$$

distribution of the m numerical value points in each sub-interval is determined; the number of the numerical value points contained in each sub-interval is counted, respectively; and the number Cₖ of the dispersed droplets containing k (k=0, 1, 2, . . . , m) numerical value points is counted.

4. The estimated valueM of the total number of the target molecules in all dispersed droplets is calculated according to C₀ and the above analysis and calculation method. Each time when m and the variation coefficient of the corresponding volume are determined, one M will be generated. This process is repeated for 500 times, and a mean and a standard deviation of the 500 Ms are calculated.

5. According to the above simulation data, linear fitting is performed on all the m values and the mean of the corresponding Ms under the condition of the same variation coefficient of the volume, and an error bar is marked. In the same coordinate system, the fitting curves corresponding to variation coefficients of different volumes are superimposed to analyze and evaluate the influence of the variation of the volumes of the dispersed droplets on the absolute quantitative precision, accuracy, dynamic range, and the like.

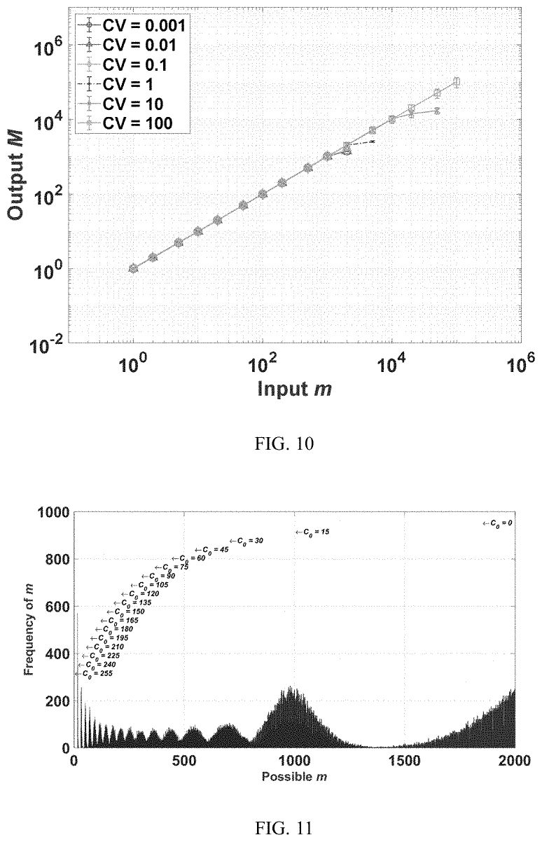

The visualization results of the simulation experiment are shown in FIG. 10. There are 6 fitting curves in the figure, respectively corresponding to fitting results when the variation coefficients of the volumes are 0.001, 0.01, 0.1, 1, 10 and 100. The data in the figure show that the variation of the volume has a greater impact on the quantitative results, and a greater variation of the volumes leads to a wider dynamic range. When the variation coefficient is 100, only 256 dispersed droplets can be used to accurately quantify 100,000 target molecules.

Embodiment 3 of verifying the simulation calculation method: In this embodiment, the random simulation method of the present disclosure is used to analyze a mapping relationship between the total number C₀ of the zones or droplets that do not contain the target molecules and the total number m of the target molecules, and thus possible ranges of m and the most likely value of M are calculated.

1. Values of the preset number m of molecules is all integers from 1 to 2001, namely 1, 2, 3, . . . , 2000, 2001.

2. The number n of the dispersed droplets generated by the random emulsification is preset to be 256, and the logarithmic Gaussian distribution is set (the volumes of the droplets generated by random emulsification in general case all satisfy the logarithmic Gaussian distribution), wherein the compliance mean of the volumes νᵢ of the dispersed droplets is 4. The standard deviations of the volumes of the dispersed droplets are respectively set to be 4, and the corresponding variation coefficients are set to be 1. 256 volume numerical values are randomly generated according to the determined parameters, and the volume parameters are kept unchanged for each subsequent simulation.

3. The sum

$${\sum }_{i = 1}^{n} {v}_{i}$$

of the volumes of the 256 dispersed droplets is calculated, and the above m numerical value points are randomly generated at the interval of

$$[ 0 , {{\sum }_{i = 1}}^{n} {v}_{i} ] ;$$

according to sub-intervals

$$[ {{\sum }_{t = 1}}^{i} {v}_{t} - {v}_{i} , {{\sum }_{t = 1}}^{i} {v}_{t} ]$$

corresponding to the 256 dispersed droplets in the interval

$$[ 0 , {{\sum }_{i = 1}}^{n} {v}_{i} ]$$

distribution of the m numerical value points in each sub-interval is determined; the number of the numerical value points contained in each sub-interval is counted, respectively; and the number Cₖ of the dispersed droplets containing k (0≤k≤500) numerical value points is counted.

4. Each time when the preset m value adopts a numerical value from all the integers from 1 to 2001 (the 256 volume numerical values are kept unchanged), the above steps are repeated for 1000 times. A pair of mappings of one m and a result C₀ can be obtained in each repetition. When all 1000×2001 random simulation experiments are completed, a total of 1000×2001 pairs of correspondence relationships between the preset m numerical value and the C₀ numerical value can be obtained. The preset m values corresponding to the same C₀ value are classified and sorted; all possible values of m are counted; and the frequency of each value of m is calculated. The counted results are superimposed in the same coordinate system, and possible ranges of m and the most likely value of M are calculated and analyzed based on actual experimental data.

The visualization results based on the simulation calculation method are shown in the multi-peak histogram in FIG. 11, wherein the horizontal axis of the coordinate system represents the possible ranges of m, and the vertical axis of the coordinate system is the frequency of each m. Each peak in the figure represents statistical results of all the possible preset values of m corresponding to a certain C₀ numerical value. The C₀ numerical values from left to right are 255, 240, 225, 210, 195, 180, 165, 150, 135, 120, 105, 90, 75, 60, 45, 30, 15, and 0. With the decrease of the above C₀ numerical value, the corresponding possible value of m increases, and its range also becomes larger, indicating a decrease in the C₀ numerical value, which will cause the uncertainty of the value of M needed to be calculated to become larger, thereby affecting the absolute quantitative precision. In addition, the probability density function of the m values can be calculated by fitting or interpolation according to frequency data points of the m values contained in each single peak, so as to calculate the result E(m) of the simulation calculation method and the confidence interval [mₘᵢₙ, mₘₐₓ] corresponding to m, namely a calculation result and confidence interval of the total number of molecules obtained in this simulation method.

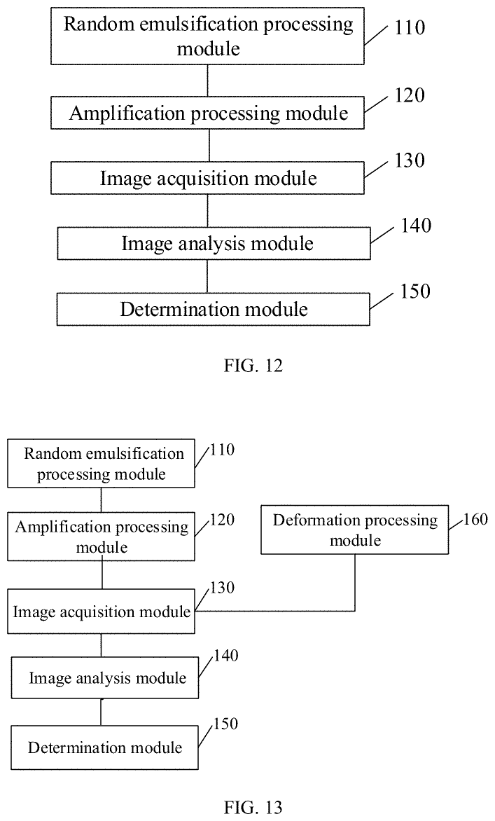

FIG. 12 is a structural schematic diagram of a random emulsification digital absolute quantitative analysis device provided in an embodiment of the present disclosure.

As shown in FIG. 12, the random emulsification digital absolute quantitative analysis device includes a random emulsification processing module 110, an amplification processing module 120, an image acquisition module 130, an image analysis module 140, and a determination module 150.

The random emulsification processing module 110 is configured to perform random emulsification processing on a system to be emulsified in a preset container to obtain several isolated reaction zones or droplets, wherein the system to be emulsified includes a sample to be tested; the total number of the reaction zones or droplets is randomly generated; the total number is a positive integer greater than 1; the reaction zones or droplets are randomly generated; and a volume of each zone or droplet is randomly (or arbitrarily)generated, and a sum of the volumes is not greater than a volume of the emulsified system.

The amplification processing module 120 is configured to perform amplification processing on the reaction zones or droplets.

The image acquisition module 130 is configured to, in response to detecting that the amplification ends, acquire images of the reaction zones or droplets to obtain a target image.

The image analysis module 140 is configured to analyze image regions, corresponding to the respective reaction zones or droplets, in the target image to obtain volume information of the respective reaction zones or droplets; determine presence of target molecules to be tested in the reaction zones or droplets; and count the number of reaction zones or droplets that do not contain the target molecules.

The determination module 150 is configured to determine, based on the total number of the reaction zones or droplets, the volume information of the respective reaction zones or droplets, the presence of the target molecules to be tested in the reaction zones or droplets and the number of the reaction zones or droplets that do not contain the target molecules, the total number of the target molecules in the sample to be tested.

In one embodiment of the present disclosure, for facilitating the subsequent rapid analysis, on the basis of the target image, of the volume information of the respective reaction zones or droplets, the presence of the target molecules to be tested in the zones or droplets and the number of the reaction zones or droplets that do not contain the target molecules, based on the device embodiment shown in FIG. 12, as shown in FIG. 13, the device may further include:

a deformation processing module 160 configured to perform squeezing deformation processing on each amplified reaction zone or droplet.

In one embodiment of the present disclosure, the image analysis module 140 is specifically configured to extract features of the image regions, corresponding to the respective reaction zones or droplets, in the target image, to obtain feature information corresponding to each image region; for each image region, match the feature information of the image region with preset feature information; in response to that the feature information of the image region is not matched with the preset feature information, determine that the reaction zone or droplet corresponding to the image region does not contain the target molecules; and determine the total number of image regions, which are not matched with the preset feature information, in the target image, and taking the total number of the image regions as the number of the reaction zones or droplets that do not contain the target molecules.

In one embodiment of the present application, the number of the target molecules in the respective reaction zones or droplets complies with the Poisson distribution of independently non-identical distribution, and the number of the reaction zones or droplets that do not contain the target molecules complies with the Poisson binomial distribution. The total number of the target molecules in the sample to be tested is determined according to the following formula:

$${{\sum }_{p = 1}}^{n - j} \frac{{v}_{p} \times {e}^{- {mv}_{p} / {{\sum {v}_{i}}_{i = 1}}^{n}}}{{{\sum }_{i = 1}}^{n} {v}_{i} \times ( 1 - {e}^{- {mv}_{p} / {{\sum {v}_{i}}_{i = 1}}^{n}} )} = {{\sum }_{q = 1}}^{j} ( {v}_{q} / {{\sum }_{i = 1}}^{n} {v}_{i} )$$

wherein m represents the total number of the target molecules to be determined in the emulsified system; n represents the total number of the reaction zones or droplets; j represents a value of the number C₀ of the reaction zones or droplets that do not contain the target molecules; νᵢ(i=1, 2 , . . . , n) represents the volume of an i^{th} reaction zone or droplet; νₚ(p=1, 2, 3, . . . , n−j) represents the volume of a p^{th} reaction zone or droplet that contains the target molecules; ν_{q}(q=1, 2, 3, . . . , j) represents the volume of a q^{th} reaction zone or droplet that does not contain the target molecules; and e is a natural constant. As described above n, j, vᵢ, vₚ, and v_{q} are all determined or statistically obtained by analyzing the target image.

In one embodiment of the present disclosure, the preset amplification system includes a preset indicator. During the amplification processing on the reaction zones or droplets, in response to detecting that an intensity of an indication signal of the preset indicator no longer changes, it is determined that the amplification processing ends.

It should be noted that the explanation of the above random emulsification digital absolute quantitative analysis method embodiment is also applicable to the random emulsification digital absolute quantitative analysis device of this embodiment, which will not be repeated here.

According to the random emulsification digital absolute quantitative analysis device provided by the embodiment of the present disclosure, random emulsification processing is performed on a system to be emulsified in a preset container to obtain several isolated reaction zones or droplets, and amplification processing is performed on the reaction zones or droplets; In response to detecting that the amplification processing ends, images of the amplified reaction zones or droplets are acquired to obtain a target image; image regions, corresponding to the respective reaction zones or droplets, in the target image are analyzed to obtain volume information of the respective reaction zones or droplets, and presence of target molecules to be tested in the reaction zones or droplets is determined; the number of reaction zones or droplets that do not contain the target molecules is counted; and the total number of the target molecules in the sample to be tested is determined based on the volume information and the preset number of the respective reaction zones or droplets and the number of the reaction zones or droplets that do not contain the target molecules. Thus, the total number of the target molecules in the sample to be tested is accurately determined, which facilitates a requirement of an absolute quantitative analysis of a sample to be tested at any concentration.

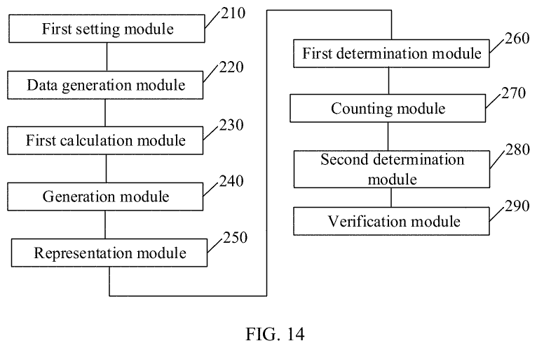

FIG. 14 is a structural schematic diagram of a simulation system provided in an embodiment of the present disclosure. It should be noted that the simulation system is configured to simulate formation of a zone with any size or dispersed droplets with any volume for achieving calculation of digital absolute quantitative testing.

As shown in FIG. 14, the simulation system includes:

a first setting module 210 configured to set the total number of target molecules to be m, wherein m is an integer greater than or equal to 0;

a data generation module 220 configured to set the total number of reaction zones or droplets to be n, and generate, based on the set total number n of the reaction zones or droplets, volume values νᵢ respectively corresponding to the n reaction zones or n droplets, wherein νᵢ represents a volume value of an i^{th} reaction zone or droplet, i=1, 2, 3, . . . , n, wherein n is an integer greater than 1;

a first calculation module 230 configured to calculate, based on the volume values corresponding to the n reaction zones or the n droplets, the total volume

$${{\sum }_{i = 1}}^{n} {v}_{i}$$

of the fluid system to be quantified;

a generation module 240 configured to randomly generate, based on the total volume of the fluid system to be quantified, m groups of coordinate numerical value sets, wherein a range of elements in the coordinate numerical value sets does not exceed the total volume of the fluid system to be quantified;

a representation module 250 configured to represent, based on a dimension of each coordinate numerical value set, the volume value of each reaction zone or droplet as n numerical value intervals which have the dimension and are connected according to a preset sequence;

a first determination module 260 configured to determine the number X, of coordinate numerical values contained in each of the n numerical value intervals;

a counting module 270 configured to count the total number of numerical value intervals containing zero coordinate numerical value, and take the obtained total number as the number C₀ of reaction zones or droplets that do not contain target molecules;

a second determination module 280 configured to determine, based on the total volume

$${{\sum }_{i = 1}}^{n} {v}_{i}$$

of the fluid system to be quantified, the total number n of the reaction zones or droplets, the volume value νᵢ of the respective reaction zones or droplets and the number C₀ of the reaction zones or droplets that do not contain the target molecules, an estimated value M of the total number of the target molecules; and

a verification module 290 configured to compare whether the set total number m of the target molecules and the estimated value M of the total number of the target molecules are within a preset error range; and in response to being within the preset error range, determine that the simulation system is capable of performing the calculation of digital absolute quantitative testing.

In one embodiment of the present disclosure, the device may further include:

a second setting module configured to set parameter information of a preset distribution with which the volumes of the reaction zones or droplets comply;

a data generation module 220 specifically configured to generate based on the parameter information of the preset distribution and the set total number n of the reaction zones or droplets, the volume values respectively corresponding to the n reaction zones or n droplets.

The preset distribution may include, but is not limited to, Gaussian distribution, logarithmic Gaussian distribution and uniform distribution, and the parameter information includes a mean, a standard deviation and a variation coefficient.

It should be noted that the foregoing explanation of the method embodiment can also be applicable to the simulation system of this embodiment, which will not be repeated in this embodiment.

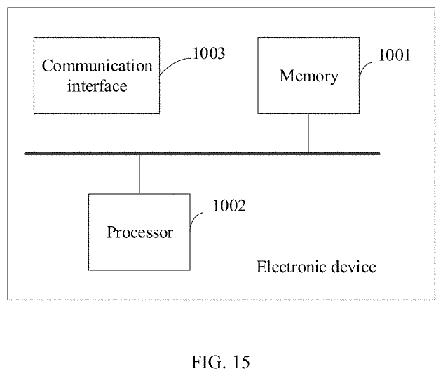

FIG. 15 is a structural schematic diagram of an electronic device provided in an embodiment of the present disclosure. The electronic device includes:

a memory 1001, a processor 1002, and a computer program stored in the memory 1001 and executable on the processor 1002.

The processor 1002 executes the program to implement the random emulsification digital absolute quantitative analysis method provided in the above embodiment, or to implement the calculation method, provided in the above embodiment, for achieving digital absolute quantitative testing by simulating formation of a zone with any size or dispersed droplets with any volume.

Further, the electronic device also includes:

a communication interface 1003 configured for communication between the memory 1001 and the processor 1002.

The memory 1001 is configured to store computer programs that are executable in the processor 1002.

The memory 1001 may include a high-speed random-access memory (RAM), or a non-volatile memory, for example, at least one disk memory.

The processor 1002 is configured to implement the random emulsification digital absolute quantitative analysis method of the above embodiment when executing the program.

If the memory 1001, the processor 1002, and the communication interface 1003 are independently implemented, the communication interface 1003, the memory 1001, and the processor 1002 can be connected to each other through a bus and complete communication with each other. The bus may be an Industry Standard Architecture (ISA) bus, a Peripheral Component Interconnection (PCI) bus, or an Extended Industry Standard Architecture (EISA) bus, etc. The bus may be divided into an address bus, a data bus, a control bus, and the like. For ease of representation, only a thick line is used in FIG. 15, but it does not mean that there is only one bus or one type of bus.

Optionally, in terms of specific implementation, if the memory 1001, the processor 1002 and the communication interface 1003 are integrated on one chip, the memory 1001, the processor 1002 and the communication interface 1003 can communicate with each other through internal interfaces.

The processor 1002 may be a Central Processing Unit (CPU), or an Application Specific Integrated Circuit (ASIC), or is configured to implement one or more ICs of the embodiment of the present disclosure.

This embodiment also provides a computer-readable storage medium having a computer program stored thereon, characterized in that the program when being executed by a processor, implements the above-mentioned random emulsification digital absolute quantitative analysis method or the calculation method for achieving digital absolute quantitative testing by simulating formation of a zone with any size or dispersed droplets with any volume.

In the description of this specification, descriptions of the reference terms such as “one embodiment”, “some embodiments”, “examples”, “specific examples,” or “some examples” mean that specific features, structures, materials or characteristics described in combination with the embodiments or examples are included in at least one embodiment or example of the present disclosure. In this specification, the schematic representations of the above terms do not necessarily refer to the same embodiment or example. Moreover, the described specific features, structures, materials or characteristics may be combined in any one or more embodiments or examples in an appropriate manner. In addition, those skilled in the art can connect and combine the different embodiments or examples and the features of the different embodiments or examples described in this specification without contradicting each other.

In addition, the terms “first” and “second” are used for descriptive purposes only and are not to be understood to indicate or imply relative importance or to imply the number of indicated technical features. Therefore, features defined by “first” and “second” can explicitly instruct or impliedly include at least one feature. In the description of the present disclosure, unless expressly specified otherwise, the meaning of the “plurality” is at least two, such as two and three.

Any process or method description in the flow chart or described in other ways herein can be understood as a module, segment or part of a code that includes one or more executable instructions for implementing specific logical functions or steps of the process. The scope of the preferred embodiments of the present disclosure includes additional implementations, which may not be in the order shown or discussed, including performing functions in a substantially simultaneous manner or in the reverse order according to the functions involved. This should be understood by those skilled in the art to which the embodiments of the present disclosure belong.

The logic and/or steps represented in flow charts or otherwise described herein, for example, may be considered as an ordered list of executable instructions for implementing logical functions, may be specifically implemented in any computer-readable medium for use with, or in conjunction with, an instruction execution system, device, or equipment (such as a computer-based system, a system including a processor, or other system that can acquire instructions from and execute instructions from the instruction execution system, device, or equipment). In terms of this specification, a “computer-readable medium” can be any device that can contain, store, communicate, propagate, or transport the program to be used by or in conjunction with an instruction execution system, device, or equipment. More specific examples (non-exhaustive list) of computer-readable media include the following: an electrical connection part (an electronic device) with one or more wiring, a portable computer disk cartridge (a magnetic device), a random-access memory (RAM), a read only memory (ROM), an erasable editable read only memory (EPROM or flash memory), a fiber optic device, and a portable compact disc read only memory (CDROM). In addition, the computer-readable medium may even be paper or other suitable media on which the program may be printed, as optical scanning can be performed, for example, on the paper or other media, and next, editing and interpretation are performed; other suitable manners are used for performing processing if necessary, so as to obtain the program in an electronic manner; and the program is then stored in a computer memory.

It should be understood that each part of the present disclosure can be implemented by hardware, software, firmware or a combination thereof In the above implementation modes, multiple steps or methods can be implemented by software or firmware stored in a memory and executed by a suitable instruction execution system. For example, if it is implemented by hardware, as in another implementation, it can be implemented by any one or a combination of the following technologies known in the art: discrete logic circuits with logic gate circuits used to realize logic functions for data signals, application-specific integrated circuits with suitable combinational logic gate circuits, programmable gate arrays (PGAs), field programmable gate arrays (FPGAs), etc.

Those of ordinary skill in the art can understand that implementation of all or a part of the steps in the method of the foregoing embodiments can be completed by a program that instructs relevant hardware. The program may be stored in a computer-readable storage medium. The program can include one of or a combination of the steps of the method embodiment.

In addition, all functional units in all the embodiments of the present disclosure can be integrated into one processing module, or each unit can physically exist alone, or two or more units can be integrated in one module. The above integrated modules can be implemented in the form of hardware, or can be implemented in the form of software functional modules. The integrated module, if implemented in the form of a software functional unit and sold or used as a standalone product, may be stored in a computer-readable storage medium.

The above-mentioned storage medium may be a read-only memory, a magnetic disk or an optical disk, and the like. Although the embodiments of the present disclosure have been shown and described above, it can be understood that the above embodiments are exemplary and should not be construed as limiting the present disclosure. Those of ordinary skill in the art can make changes, modifications, substitutions, and variations to the above-mentioned embodiments within the scope of the present disclosure.

1. A random emulsification digital absolute quantitative analysis method, the method comprising:
performing random emulsification processing on a system to be emulsified in a preset container to obtain several isolated reaction zones or droplets, wherein the system to be emulsified comprises a sample to be tested; the total number of the reaction zones or droplets is randomly generated; the total number is a positive integer greater than 1; the reaction zones or droplets are randomly generated; and a volume of each zone or droplet is randomly generated, and a sum of the volumes is not greater than a volume of the emulsified system;
performing amplification processing on the reaction zones or droplets;
acquiring, subsequent to that the amplification processing ends, images of the reaction zones or droplets to obtain a target image;
analyzing image regions, corresponding to the respective reaction zones or droplets, in the target image to obtain volume information of the respective reaction zones or droplets; determining presence of target molecules to be tested in the reaction zones or droplets; and counting the number of reaction zones or droplets that do not contain the target molecules;
determining, based on the total number of the reaction zones or droplets, the volume information of the respective reaction zones or droplets, the presence of the target molecules to be tested in the reaction zones or droplets and the number of the reaction zones or droplets that do not contain the target molecules, the total number of the target molecules in the sample to be tested,
wherein the number of the reaction zones or droplets that do not contain the target molecules complies with the Poisson binomial distribution, and wherein the total number of the target molecules in the sample to be tested is determined according to the following formula:
$$\underline{{{\sum }_{p = 1}}^{n - j} \frac{{v}_{p} \times {e}^{- {mv}_{p} / {{\sum {v}_{i}}_{i = 1}}^{n}}}{{{\sum }_{i = 1}}^{n} {v}_{i} \times ( 1 - {e}^{- {mv}_{p} / {{\sum {v}_{i}}_{i = 1}}^{n}} )} = {{\sum }_{q = 1}}^{j} ( {v}_{q} / {{\sum }_{i = 1}}^{n} {v}_{i} )}$$
wherein m represents the total number of the target molecules to be determined in the emulsified system; n represents the total number of the reaction zones or droplets; j represents a value of the number C₀ of the reaction zones or droplets that do not contain the target molecules; vᵢ(i=1, 2 , 3, . . . , n) represents the volume of an i^{th} reaction zone or droplet; vₚ(p=1, 2, 3, . . . , n−j) represents the volume of a p^{th} reaction zone or droplet that contains the target molecules; v_{q} (q=1, 2, 3, . . . , j) represents the volume of a q^{th} reaction zone or droplet that does not contain the target molecules; and e is a natural constant.

2. The method according to claim 1, further comprising, prior to acquiring images of the reaction zones or droplets to obtain a target image to be analyzed:
performing squeezing deformation processing on each amplified reaction zone or droplet.

3. The method according to claim 1, wherein said analyzing image regions, corresponding to the respective reaction zones or droplets, in the target image, and counting the number of reaction zones or droplets that do not contain the target molecules comprises:
extracting features of the image regions, corresponding to the respective reaction zones or droplets, in the target image, to obtain feature information corresponding to each image region;
for each image region, matching the feature information of the image region with preset feature information; in response to that the feature information of the image region is not matched with the preset feature information, determining that the reaction zone or droplet corresponding to the image region does not contain the target molecules; and
determining the total number of image regions, which are not matched with the preset feature information, in the target image, and taking the total number of the image regions as the number of the reaction zones or droplets that do not contain the target molecules.

4. (canceled)

5. The method according to claim 1, wherein the preset amplification system comprises a preset indicator; during the amplification processing on the reaction zones or droplets, in response to detecting that an intensity of an indication signal of the preset indicator is no longer to change, it is determined that the amplification processing ends.

6. A simulation method for simulating formation of a zone with any size or dispersed droplets with any volume to achieve calculation of digital absolute quantitative testing, the method being applied to a simulation system and comprising:
setting the total number of target molecules to be m, wherein m is an integer greater than or equal to 0;
setting the total number of reaction zones or droplets to be n, and generating, based on the set total number n of the reaction zones or droplets, volume values v ᵢ respectively corresponding to the n reaction zones or n droplets, wherein νᵢ represents a volume value of an i^{th} reaction zone or droplet, I=1, 2, 3, . . . , n, wherein n is an integer greater than 1;
calculating a total volume
$${{\sum }_{i = 1}}^{n} {v}_{i}$$
of a fluid system to be quantified based on the volume values respectively corresponding to the n reaction zones or n droplets;
randomly generating m groups of coordinate numerical value sets based on the total volume of the fluid system to be quantified, wherein a range of elements in the coordinate numerical value sets does not exceed the total volume
$${{\sum }_{i = 1}}^{n} {v}_{i}$$
of the fluid system to be quantified;
representing, based on a dimension of each coordinate numerical value set, the volume value of each reaction zone or droplet as n numerical value intervals which have the dimension and are connected according to a preset sequence;
determining the number of coordinate numerical values contained in each of the n numerical value intervals;
counting the total number of numerical value intervals containing zero coordinate numerical value, and taking the obtained total number as the number C₀ of reaction zones or droplets that do not contain target molecules;
determining, based on the total volume
$${{\sum }_{i = 1}}^{n} {v}_{i}$$
of the fluid system to be quantified, the total number n of the reaction zones or droplets, the volume value vᵢ of the respective reaction zones or droplets and the number C₀ of the reaction zones or droplets that do not contain the target molecules, an estimated value M of the total number of the target molecules; and
comparing whether the set total number m of the target molecules and the estimated value M of the total number of the target molecules are within a preset error range; and in response to being within the preset error range, determining that the simulation system is capable of performing calculation of digital absolute quantitative testing.

7. The method according to claim 6, further comprising:
setting parameter information of a preset distribution with which the volumes of the reaction zones or droplets comply,
wherein said generating, based on the set total number n of the reaction zones or droplets, volume values respectively corresponding to the n reaction zones or n droplets comprises:
generating, based on the parameter information of the preset distribution and the set total number n of the reaction zones or droplets, the volume values respectively corresponding to the n reaction zones or n droplets.

8. The method according to claim 7, wherein the preset distribution comprises Gaussian distribution, logarithmic Gaussian distribution, and uniform distribution, and wherein the parameter information comprises a mean, a standard deviation, and a variation coefficient.

9. A random emulsification digital absolute quantitative analysis device, the device comprising:
a random emulsification processing module configured to perform random emulsification processing on a system to be emulsified in a preset container to obtain several isolated reaction zones or droplets, wherein the system to be emulsified comprises a sample to be tested; the total number of the reaction zones or droplets is randomly generated; the total number is a positive integer greater than 1; the reaction zones or droplets are randomly generated; and a volume of each zone or droplet is randomly generated, and a sum of the volumes is not greater than a volume of the emulsified system;
an amplification processing module configured to perform amplification processing on the reaction zones or droplets;
an image acquisition module configured to, in response to detecting that the amplification ends, acquire images of the reaction zones or droplets to obtain a target image;
an image analysis module configured to analyze image regions, corresponding to the respective reaction zones or droplets, in the target image to obtain volume information of the respective reaction zones or droplets; determine presence of target molecules to be tested in the reaction zones or droplets; and count the number of reaction zones or droplets that do not contain the target molecules; and
a determination module configured to determine, based on the total number of the reaction zones or droplets, the volume information of the respective reaction zones or droplets, the presence of the target molecules to be tested in the reaction zones or droplets and the number of the reaction zones or droplets that do not contain the target molecules, the total number of the target molecules in the sample to be tested
wherein the number of the reaction zones or droplets that do not contain the target molecules complies with the Poisson binomial distribution, and wherein the total number of the target molecules in the sample to be tested is determined according to the following formula:
$$\underline{{{\sum }_{p = 1}}^{n - j} \frac{{v}_{p} \times {e}^{- {mv}_{p} / {{\sum {v}_{i}}_{i = 1}}^{n}}}{{{\sum }_{i = 1}}^{n} {v}_{i} \times ( 1 - {e}^{- {mv}_{p} / {{\sum {v}_{i}}_{i = 1}}^{n}} )} = {{\sum }_{q = 1}}^{j} ( {v}_{q} / {{\sum }_{i = 1}}^{n} {v}_{i} )}$$
wherein m represents the total number of the target molecules to be determined in the e emulsified system; n represents the total number of the reaction zones or droplets; j represents a value of the number C₀ of the reaction zones or droplets that do not contain the target molecules; vᵢ(i=1, 2 , 3, . . . , n) represents the volume of an i^{th} reaction zone or droplet; vₚ(p=1, 2, 3, . . . , n−j) represents the volume of a p^{th} reaction zone or droplet that contains the target molecules; v_{q}(q=1, 2, 3, . . . , j) represents the volume of a q^{th} reaction zone or droplet that does not contain the target molecules; and e is a natural constant.

10. The device according to claim 9, further comprising:
a deformation processing module configured to perform squeezing deformation processing on each amplified reaction zone or droplet.

11. The device according to claim 9, wherein the image analysis module is configured to:
extract features of the image regions, corresponding to the respective reaction zones or droplets, in the target image, to obtain feature information corresponding to each image region;
for each image region, match the feature information of the image region with preset feature information; in response to that the feature information of the image region is not matched with the preset feature information, determine that the reaction zone or droplet corresponding to the image region does not contain the target molecules; and
determine the total number of image regions, which are not matched with the preset feature information, in the target image, and taking the total number of the image regions as the number of the reaction zones or droplets that do not contain the target molecules.

12. (canceled)

13. The device according to claim 9, wherein the preset amplification system comprises a preset indicator; during the amplification processing on the reaction zones or droplets, in response to detecting that an intensity of an indication signal of the preset indicator no longer changes, it is determined that the amplification processing ends.

14. A simulation system, configured to simulate formation of a zone with any size or dispersed droplets with any volume for achieving calculation of digital absolute quantitative testing, the simulation system comprising:
a first setting module configured to set the total number of target molecules to be m, wherein m is an integer greater than or equal to 0;
a data generation module configured to set the total number of reaction zones or droplets to be n, and to generate, based on the set total number n of the reaction zones or droplets, volume values vᵢ respectively corresponding to the n reaction zones or n droplets, wherein νᵢ represents a volume value of an i^{th} reaction zone or droplet, i=1, 2, 3, . . . , n, wherein n is an integer greater than 1;
a first calculation module configured to calculate, based on the volume values respectively corresponding to the n reaction zones or the n droplets, the total volume
$${{\sum }_{i = 1}}^{n} {v}_{i}$$
of the fluid system to be quantified;
a generation module configured to randomly generate, based on the total volume
$${{\sum }_{i = 1}}^{n} {v}_{i}$$
of the fluid system to be quantified, m groups of coordinate numerical value sets, wherein a range of elements in the coordinate numerical value sets does not exceed the total volume of the fluid system to be quantified;
a representation module configured to represent, based on a dimension of each coordinate numerical value set, the volume value of each reaction zone or droplet as n numerical value intervals which have the dimension and are connected according to a preset sequence;
a first determination module configured to determine the number of coordinate numerical values contained in each of the n numerical value intervals;
a counting module configured to count the total number of numerical value intervals containing zero coordinate numerical value, and take the obtained total number as the number C₀ of reaction zones or droplets that do not contain target molecules;
a second determination module configured to calculate, based on the total volume
$${{\sum }_{i = 1}}^{n} {v}_{i}$$
of the fluid system to be quantified, the total number n of the reaction zones or droplets, the volume value vᵢ of the respective reaction zones or droplets and the number Co of the reaction zones or droplets that do not contain the target molecules, an estimated value M of the total number of the target molecules; and
a verification module configured to compare whether the set total number m of the target molecules and the estimated value M of the total number of the target molecules are within a preset error range; and in response to being within the preset error range, determine that the simulation system is capable of performing the calculation of digital absolute quantitative testing.

15. The simulation system according to claim 14, further comprising:
a second setting module configured to set parameter information of a preset distribution with which the volumes of the reaction zones or droplets comply,
wherein the data generation module is specifically configured to:
generate, based on the parameter information of the preset distribution and the set total number n of the reaction zones or droplets, the volume values respectively corresponding to the n reaction zones or n droplets.

16. The simulation system according to claim 15, wherein the preset distribution comprises, Gaussian distribution, logarithmic Gaussian distribution, and uniform distribution, and wherein the parameter information comprises a mean, a standard deviation, and a variation coefficient.

17. An electronic device, comprising:
a memory having a computer program stored thereon and executable on the processor; and
a processor,
wherein the processor executes the program to implement the random emulsification digital absolute quantitative analysis method according to claim 1.

18. A computer-readable storage medium, having a computer program stored thereon, wherein the program, when being executed by a processor, implements the random emulsification digital absolute quantitative analysis method according to claim 1.

19. An electronic device, comprising:
a memory having a computer program stored thereon and executable on the processor; and
a processor,
wherein the processor executes the program to implement the calculation method for achieving digital absolute quantitative testing by simulating formation of a zone with any size or dispersed droplets with any volume according to claim 6.

20. A computer-readable storage medium, having a computer program stored thereon, wherein the program, when being executed by a processor, implements the calculation method for achieving digital absolute quantitative testing by simulating formation of a zone with any size or dispersed droplets with any volume according to claim 6.
